(19) United States
(12) Patent Application Publication
(10) Pub. No.: US 2010/0241219 A1
(43) Pub. Date: Sep. 23, 2010

(54) MEDICAMENT DELIVERY ARTICLE, ACCESSORY & SYSTEM

(75) Inventors: Lloyd K. Willard, Miltona, MN (US); Mark J. Whalen, Alexandria, MN (US); John M. Reid, Garfield, MN (US); Chad S. Smith, Parkers Prairie, MN (US); Charles L. Euteneuer, St. Michael, MN (US); Paul Buscemi, Long Lake, MN (US)

Correspondence Address: NAWROCKI, ROONEY & SIVERTSON;SUITE 401, BROADWAY PLACE EAST, 3433 BROADWAY STREET NORTHEAST, MINNEAPOLIS, MN, 554133009, US

(73) Assignee: ABBEYMOOR MEDICAL , INC, Parkers Prairie, MN (US)

(21) Appl. No.: 11/993,394

(22) PCT Filed: Jun. 20, 2006
(86) PCT No.: PCT/US06/23806

§ 371 Date: Jun. 9, 2010

Related U.S. Application Data
(60) Provisional application No. 60/691,635, filed on Jun. 20, 2005.
(60) Provisional application No. 60/691,636, filed on Jun. 20, 2005.

(51) Int. Cl. A61K 9/14; A61K 31/517; A61K 31/27; A61K 31/435; A61K 31/18; A61K 31/4985; A61K 31/496; A61K 31/704; A61K 31/337; A61M 25/00; A61F 2/82

(52) U.S. Cl. 623/1.42; 424/486; 514/266.24; 514/478; 514/284; 514/603; 514/250; 514/252.16; 514/34; 514/449; 604/523

(57) ABSTRACT

An adjunctive accessory article (25) for controlled release of at least a single physiologically active agent form an indwelling medical device so equipped is provided. The article is configurable as a tubular element upon a portion of the indwelling medical device, with barriers or facilitating coverings overlaying the carrier in furtherance of the controlled release of the active agent. To the extent that requires, affixation means for stable placement upon an indwelling device are further contemplated. Finally, a variety of application systems are described in connection to deployment of sleeve articles upon a portion of an indwelling device.

This is an international application filed under 35 USC §363, claiming priority under 35 USC §119(e) of U.S. Prov. Appl. Nos. 60/691,635 and 60/691,636, each filed Jun. 20, 2005 and each incorporated herein by reference in its entirety.

### TECHNICAL FIELD

The present invention generally relates to medical devices, more particularly to medicament delivery articles associated therewith, and for use in connection thereto, more particularly still, to medicament delivery articles for endourethral devices and the like for localized delivery of a physiologically active agent to an interior cavity to the human body, namely, to the urinary urogenital system, and yet more particularly to the lower urogenital system, inclusive of the bladder, urethra, prostate, penis, seminal vesicles, ejaculatory ducts, glands, and testes; for symptomatic relief, infection and disease treatment.

### BACKGROUND OF THE INVENTION

There are a variety of afflictions, conditions, etc. of and/or associated with human anatomy systems, structures and physiology thereof that may benefit from localized delivery of medicaments, i.e., physiologically active agents. A brief general preliminary discussion will benefit the reader in furtherance of understanding and appreciating unmet clinical needs in relation to, or in the context of, those afflictions, conditions, etc. associated with the urogenital system, more particularly, to/with the urogenital system of the human male.

Up to two million office visits annually in the United States are attributed to patients being bothered by some form of lower urinary tract symptoms (LOTS), with bladder outlet obstructions (BOO) being a major subgroup of LOTS. In men between the ages of 55 and 75 years, it is estimated that between 50 and 75% have some degree of bladder outlet obstruction, however, it may not be responsible for their symptoms.

Problems range in severity from minor inconvenience, which lowers the quality of life, to life threatening disease. The following categories of medical conditions are generally noted: (1) bladder, or more generally, lower urinary tract dysfunction; (2) infection of the urinary tract; (3) infection and disease of the reproductive organs and glands; and, (4) life threatening disease of the urogenital tract. In connection to the first category, numerous indwelling devices are well know, with physiological endourethral device considerations being numerous, well documented, and generally beyond the scope of the subject disclosure. Be that as it may, the following teachings in this area are illustrative, non-limiting, and each, in its entirety, incorporated herein by reference in furtherance of supplementing an understanding of the subject discussion: co-pending application Ser. No. ______ entitled SELF-ADJUSTING ENDOURETHRAL DEVICE & METHODS OF USE filed Jun. 20, 2006; published U.S. published patent application Pub. No. US 2002/0107540 A1; and, and issued U.S. Pat. No. 6,991,596 B2.

The previously noted categories of medical conditions are known to benefit from localized delivery of medicaments. For instance, urinary tract infections are commonly treated with prophylactic antibiotics, with slow urination or retention episodes oftentimes warranting medicaments that are used to ease voiding difficulty, normally selected from of the following pharmacological families: (1) alpha blockers; (2) alpha inhibitors; and, (3) enzyme inhibitors, e.g., finasterides, each having its own therapy mechanism, and each generally only marginally effective in relieving symptoms.

To the extent that the patient cannot accept the symptoms that remain post treatment, or if he has concern about lower urinary tract dysfunction and/or disease, urological consultation may be necessitated. As should be readily appreciated, treatment will vary greatly patient to patient, and urologist to urologist, according to the presenting conditions. Diagnosis of benign disease presents a first array of treatment possibilities, and diagnosis of malignancy a second array of treatment possibilities.

In connection to malignancy, scientific articles speculate that more than half of men over fifty years of age have some cancer cells in their prostate. Like most cancers, the treatment response depends upon the state or condition of the disease, i.e., the quality or character of advancement of the disease. Early stage prostate cancer is defined as the cells being entirely encapsulated within the prostate capsule. Prostate cancer is an unusual cancer because it can remain in the prostate capsule for as long as twenty to thirty years. When this occurs, the patient may be asymptomatic, not knowing of the disease's presence. The capsule may however, enlarge and produce similar voiding symptoms to those associated with, benign enlargement.

When the patient chooses to be screened, and cancer is detected in the prostate gland through biopsy, an assessment of disease state, i.e., generally stages one through four and variants thereof, is had. The first stage (T1) is defined or characterized as the presence of a clinically unapparent tumor, not detected by digital rectal exam (DRE), nor visible by imaging, e.g., transrectal ultrasound (TRUS). The second stage (T2) is defined or characterized as the tumor confined within the prostate, detectable by DRE, not visible on TRUS. The third stage (T3) is defined or characterized as the tumor having extending through the capsule, but not having spread to other organs. The fourth and final stage (T4) is described as the tumor being fixed or invasive of adjacent structures, other than the seminal vesicles. As their description indicates, the later stages are exceedingly serious, with patients typically undergoing external beam radiation, or systemic chemotherapy. The prostate may or may not be removed in conjunction with this treatment.

To the extent cancer is detected within the T1 or T2 stage, the prostate is likely to be either removed by a radical prostatectomy procedure, or treated in place with a minimally invasive therapy such as cryotherapy which involves freezing the prostate gland, or select localized tissue therein, or brachytherapy which attempts to kill the malignant tumor(s) in place by surgically implanting radioactive seeds into the interior of the prostate near the tumor(s). These seeds decrease their radioactivity over time due to the short half-life of the materials selected, and are intended to remain fixed in the prostate, however, movement of seeds, as far away as the lung, have been documented.

Prostate surgery, and even minimally invasive procedures, inherently include, or have associated therewith, risks that may affect the quality of life following recovery, namely, for example, incontinence and/or impotence. The sphincter mechanism may be rendered incompetent, or the nerve network central in initiating and sustaining an erection of the penis damaged.

Tables I & II herewith list and describe a variety of pharmaceutical agents which are being prescribed to treat physical conditions relating to the urological and reproductive systems. These include, without limitation, prostate disease, bladder & urinary tract infection (UTI), BPH, bladder insufficiency, cancer of the bladder, cancer of the prostate, pain, erectile dysfunction (ED), prostatitis, kidney stones, etc.

Other useful physiologically active agents include antiseptics, e.g., betadine, and local anesthetics, e.g., lydocane. Several common anesthetics used in the bladder, urethra, and vaginal areas are listed and described in Table III. Disinfecting of the mucosal membrane of the bladder, urethra or vagina require direct contact to cleanse the cavity, either prior to introduction of an indwelling device, or in situ for maintenance. Further physiologically acting agents having utility include, without limitation, antimicrobials, antibiotics, anti-inflammatories, antiseptics, hydrophilics, hydrophobics, etc.

Antimicrobial agents, e.g., silver oxides, are commonly utilized to create a surface that many bacteria are unable to, or not easily colonize. In the lower urinary-tract, this reduces the strong propensity for the initiation of urinary tract infection (UTI). Antibiotics are well known in the treatment of UTI.

Anti-inflammatory agents, non-steroidal and steroidal, are intended to reduce inflammation which may be present in benign or transition state prostate disease. Inflammation is generally present during the progression of prostate disease though its contribution to the progression is yet unconfirmed scientifically.

Antiseptics are intended to kill bacteria upon contact. Depending upon concentrations, antiseptics may be used as cleansers for physiologic maintenance of elements of the lower urinary tract, namely, the bladder and/or urethra.

Hydrophylic surfaces are generally known to act or function as sponges for water based fluids. Such surfaces are useful in delaying bacterial presence on the surface of an indwelling device when proper hydration is accomplished prior to insertion. Hydrophylic surfaces are further known to be poor surfaces for adhesion of blood constituents, e.g., proteins and platelets.

Hydrophobic surfaces repel water. Such surfaces are generally attractive to both bacteria, and blood products. Hydrophobic surfaces occasionally are proper surfaces for barriers for solution delivery.

In connection with medicament delivery systems and the like, a variety of approaches to providing a controlled release and sustained or prolonged release of a physiologically active agent are well known, see for example U.S. Pat. No. 4,601,893 (Cardinal), incorporated herein by reference in its entirety. Medicament eluting or delivery articles and/or systems have experienced a significant amount of development in recent years, specifically in two primary areas, namely, drug delivery systems providing controlled release properties, and therapeutic compounds or drugs to be used with these systems.

Therapeutic or prophylactic drugs are commonly provided to patients by delivery systems within the body. These systems may include pumps, bags, completely soluble systems, or materials containing drugs dissolved or suspended in a carrier. In a carrier approach, a medicament is intended to migrate from the carrier upon deployment to a liquid environment.

While pumps and bags are uniquely capable of providing relatively large volumes of drug, even over fairly extended periods of time, completely soluble systems can provide drugs from an element which can substantially eliminate itself over time by dissolving. The elution or migration delivery system can deliver very small amounts of drugs over an extended time period while targeting a relatively small area. Alternative delivery systems are always desirable, particularly where the delivery system can be readily adapted to fixation on many different size and shape elements and where the drug delivery element can be readily manufactured.

As noted in connection with localized solute or solution delivery, it requires that a device, element thereof, or dedicated delivery article advantageously release the solution at a preselect rate, so as to deliver a preselect effective amount of the medicament, i.e., commonly, a controlled and prolonged release of at least a single active agent to or within an ambient environment. One such delivery system approach uses controlled polymer coating technologies that provide a substrate for carrying a compound, and a cover layer that provides means to regulate the release of the compound. These systems are referred to as “carrier/barrier” systems. The compound elution or release rate can be designed based on the properties of the compound and the cover layer.

Another delivery system is predicated upon the polymeric carrier's functional characteristics, namely, its ability to be reversibly “expandable,” on a molecular level, to allow compounds to be “trapped” in interstitial molecular voids of the carrier, temporarily, and allowed to be released over a period of time when placed in the body.

Notionally, a “facilitating” element is generally understood to be an element, e.g., a layer, substratum, superstratum, laminate, lamina, etc., of the delivery article which may change its physical structure, or one or more properties thereof, e.g., as by hydration, so as to alter a capacity of the article to elute or otherwise release a medicament, e.g., from the carrier or medicament reservoir, into or to an operative environment for the article. Oftentimes, a further element, e.g., stratum, layer, lamina, etc. is further included in combination with the medicament reservoir, typically “below” or underlying same, so as to function as a pressure pump to “squeeze” the reservoir layer against a overlying element, for instance the reservoir barrier, as by “swelling” or other physical transformation. The source of the motive force may suitably consist of strata which, by their composition, can be altered to accommodate medicaments which are more/less lipophilic as the case may be. Furthermore, a series of synergistically cooperative strata or the like, which may be principally composed of urethanes but may be composed of other phase separated polymeric materials, may comprise, or be present in such articles. Stratum of such articles are known also to contain or otherwise include other phases, e.g., inorganic phases or preexisting phases such as microspheres or macrospheres, commonly 10 to 200 microns in diameter, e.g., such elements which may be encapsulated or otherwise bound to or within the medicament reservoir.

Further still, and alternately, known articles may include a series of strata or layers which may be made more/less porous via the inclusion of phase separating solvents, preferably but not necessarily, those that evaporate quickly. Such layers are commonly formed or manifest as by dipping, spraying or layering pre-made sheets into tubular structures, and may be composed of any of a variety of solvent castable polymeric materials including, but not limited to polyvinylclorides, polyethlyacrylates, polyvinylnitriles, and preferably polyurethanes, as well as curable materials such as water based latexes.

In light of the foregoing background, there thus remains a variety of unmet clinic needs for providing an article per se (i.e., an adjunctive accessory article), a mechanism by which to quickly and reliably equip an indwelling device with same, a device equipped with such article, or a device per se having integral thereto the aforedescribed functionality.

First, there remains significant need for improved introduction of solutions, more generally medicaments, into a body cavity. This is especially true in the urinary tract, and even more so in the male urinary tract, as evidenced by the prior overview of afflictions, conditions, etc. of the urogenital system.

Second, as there are a large number of physiologically active agents which are being used to treat symptoms, diseases, and/or infection within the urogenital system, an adjunctive accessory article, namely, an article discrete from an indwelling device, is believed particularly advantageous.

Third, in the course of indwelling device sterilization, e.g., as by ethylene oxide, gamma radiation, or electron beam radiation, many carrier/delivery mechanisms undergo a degree of transformation which impacts delivery efficacy. For this reason it is important to have article configuration options to prepare the solution separately from the device so as to permit specific solution selection onto a given platform, or a variety of platforms.

Fourth, the introduction of one or more solutions locally is preferable to systemic delivery, commonly via ingestion or arterial-venous access or loop, because the systemic toxicity of solutions can be extremely tough on the patient. Often the toxicities prevent the treatment, or continuum of treatment, of the infection or disease for patients with co-morbidities, or, reactions to the chemicals.

Fifth, and finally, in order to deliver the solution locally, a stable platform is needed which provides a surface with a physical presence adjacent or near the point of solution delivery for a prolonged period.

### SUMMARY OF THE INVENTION

The present invention provides an adjunctive accessory article in the form of a medicament delivery article, a mechanism by which to quickly and reliably equip an indwelling device with such article, and likewise contemplates indwelling devices so equipped, and/or a device per se having integral thereto the functionalities subsequently described. The article generally includes a polymeric active agent carrier and an active agent carried thereby. Advantageously, the article is configurable as a tubular element, or in fact may be configured as tubular element.

In addition to a carrier, the article further contemplates a barrier or facilitating element in furtherance of delivering a controlled release of at least a single physiologically active agent into a localized environment of an indwelling device. To the extent that the carrier or carrier/barrier composite/assembly requires, affixation means for stable placement upon an indwelling device are further contemplated and described.

Advantageously, but not necessarily, the accessory articles contemplated may include a bounded volume and/or voids for retaining a physiologically active solution, or a synergistically active fluid in furtherance of therapy delivery. Furthermore, the delivery of energy in furtherance of brachytherapy or the like, is enabled via the accessory article of the subject device.

In connection to mechanisms of equipping an indwelling device with the accessory article, a variety of non-limiting applicators, and thusly “systems” are provided. Three device application mechanisms or approaches are detailed for articles configured as a tubular element, namely, coil applicator, an inverted or retractable applicator, and a “slip-fit” applicator.

Additional items, advantages and features of the various aspects of the present invention will become apparent from the description of its preferred embodiments, which description should be taken in conjunction with the accompanying drawings.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles, elements and interrelationships therebetween of the invention.

FIGS. 1-5 each generally illustrate medicament delivery articles of the subject invention, more particularly, adjunctive accessory articles for application to an indwelling medical device;

FIGS. 6-8 each generally illustrate further medicament delivery articles of the subject invention, more particularly, adjunctive accessory articles having a “rolled-up” configuration for “un-rolling” onto an indwelling device, the articles generally resembling those of FIGS. 2, 3 & 5 respectively;

FIGS. 9-11 each generally illustrate medicament delivery articles of the subject invention in cooperative engagement with an indwelling medical device, more particularly, endourethral devices;

FIG. 12 illustrates yet a further embodiment of the medicament delivery article of the subject invention, more particularly, a device conforming sleeve;

FIGS. 13-15 each represent alternative sectional views of the article of FIG. 12, or more generally, the articles contemplated herein;

FIG. 16 generally depicts an article application system, more particularly, the article of FIG. 12 in combination with an applicator;

FIG. 17 illustrates a preliminary stage of article application upon an indwelling device utilizing the application system of FIG. 16;

FIG. 18 illustrates a transfer mechanism associated with the application system of FIG. 16, more particularly, a partial sectional view depicting the “unwinding” of an article holder or mandrel;

FIG. 19 illustrates, in a section view, an alternate “unwinding” configuration from that of FIG. 18;

FIG. 20(a) illustrates an alternate embodiment of an applicator for the article of, for example, FIG. 12, with FIGS. 20(b)-(g) illustrating the operability of an article receiving end portion of the applicator of FIG. 20(a);

FIG. 21 is a sectional view (alternate) of the article receiving end portion of the applicator of FIG. 20(a);

FIG. 22 depicts an alternate configuration for the applicator of FIG. 20(a);

FIG. 23(a)-(j) generally illustrates the application of the article of FIG. 12, or the like, upon the indwelling device of FIG. 9 or 10, or the like and pre-deployment, utilizing the applicator of FIG. 20(a);

FIG. 24 illustrates yet a further embodiment of the medicament delivery article of the subject invention, more particularly, adjunctive accessory article for receipt within an aperture of an indwelling device; and,

FIG. 25 illustrates the indwelling urethral device of FIG. 9 or 10 equipped with the article of FIG. 24, and an article resembling that of FIG. 5.

### DETAILED DESCRIPTION OF THE INVENTION

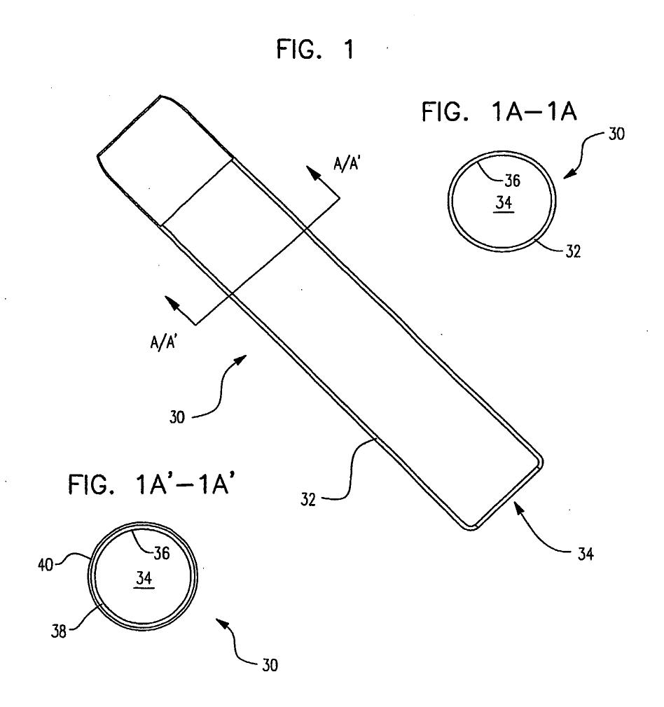

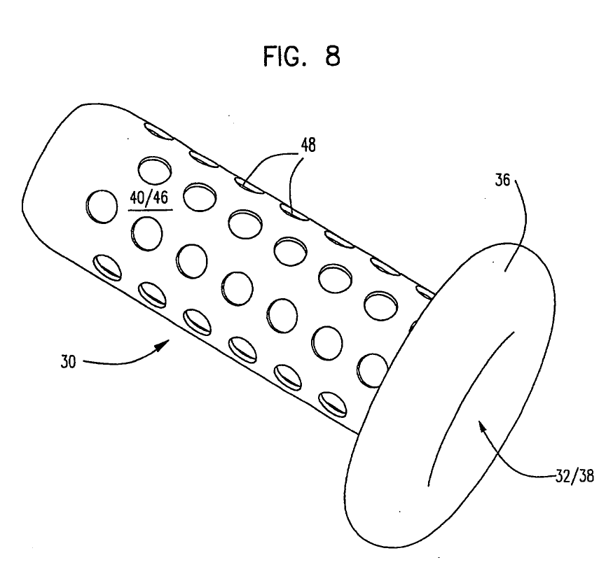

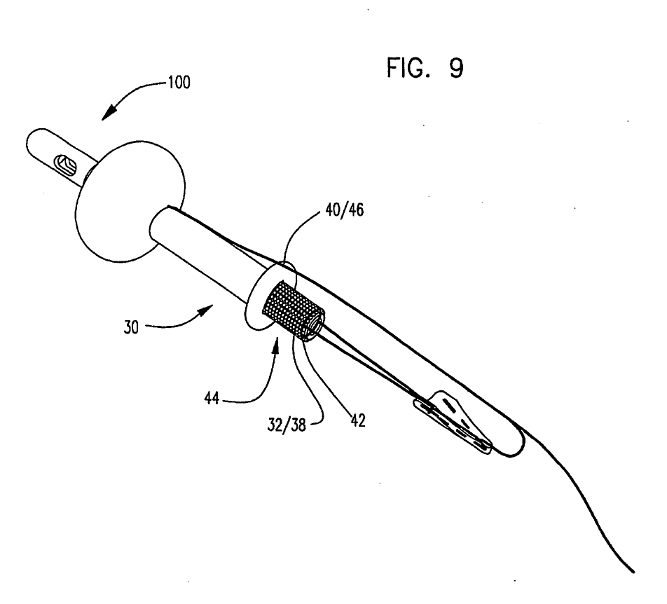

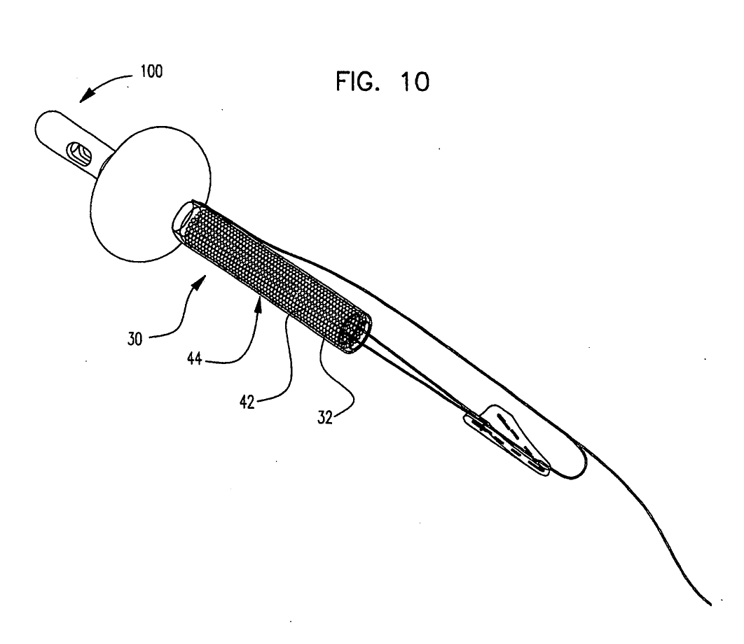

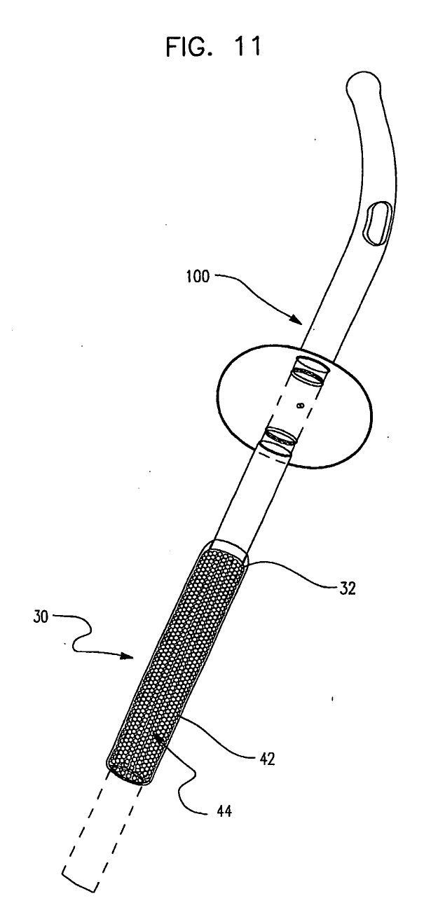

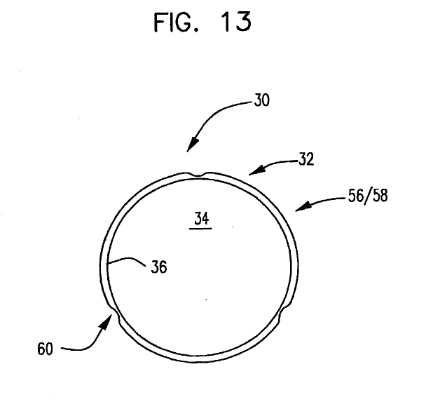

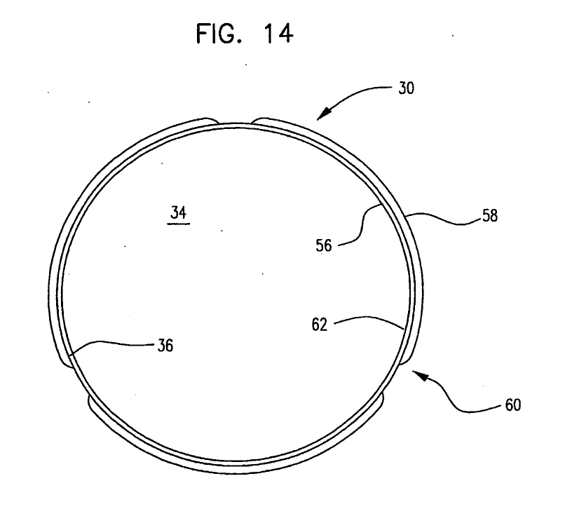

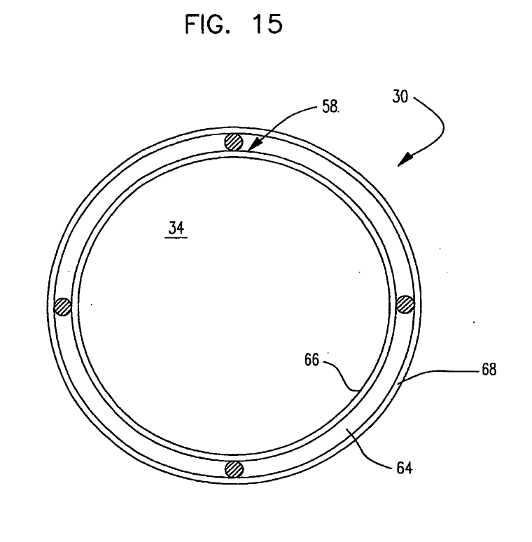

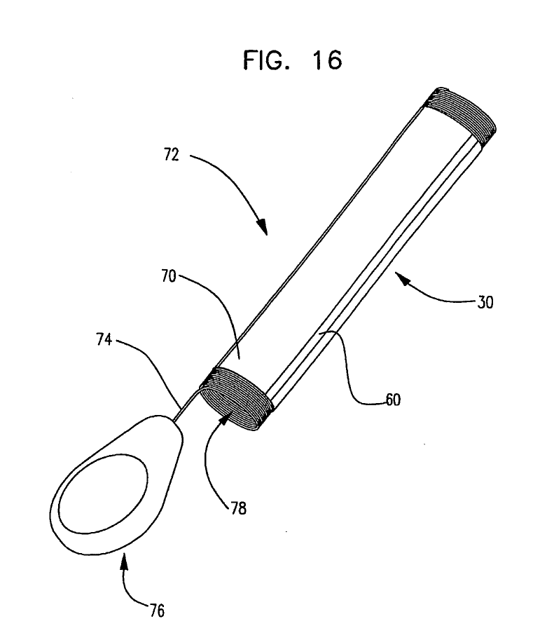

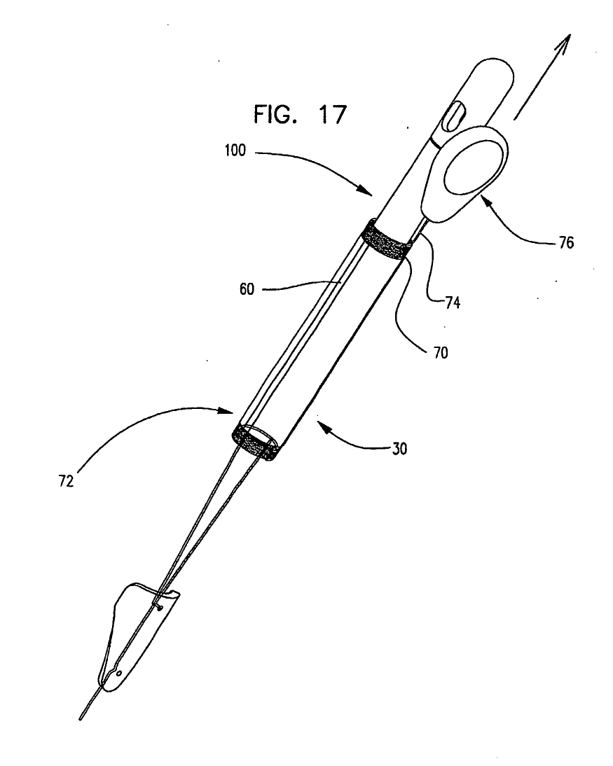

Medicament delivery articles of the subject invention, namely, adjunctive accessory articles, are generally illustrated in FIGS. 1-8, 12, and 25, with FIGS. 13-15 representing alternate sectional views of the article of FIG. 9. Indwelling medical devices, more particularly, endourethral devices, are generally illustrated in each of FIGS. 9-11, 25, and 25, and equipped, to some degree, with an adjunctive accessory article of the subject invention. The remaining figures, namely FIGS. 16-21, generally illustrate contemplated article application systems of the subject invention, more particularly, alternate applicators for equipping an indwelling medical device with select adjunctive accessory articles of the subject invention. Prior to a detailed discussion of the articles and systems of the subject invention, several preliminary or general remarks are in order.

First, the term “medicament” is to be afforded great breadth in connection to its meaning, more particularly, it is intended to embrace, among other things, notions of physiologically active agents. In-as-much as specificity may be found in the subject specification in connection with the notions of “medicament,” “agent,” etc., such specificity is intended to be illustrative and non-limiting.

Second, while emphasis is placed herein on medicament delivery articles, more particularly, articles in the nature of accessories suited for application to a wide variety of non-limiting indwelling medical devices, the features of the subject invention, and their interrelationships, need not be so limited. For instance, it is believed, and as should be readily appreciated, that one or more features, and relationships among and between same, may be integral to, or otherwise provided in an indwelling device per se.

Third, the terms “carrier” and “barrier” are generally intended as functional monikers relative to the terms medicament, agent, etc. Carrier contemplates a medium or media which includes the medicament, agent, etc. As will later be developed, the carrier may be homogenous or heterogenous, e.g., monolithic or a composite, plastic, semi-plastic or characterized as having thermoset properties. Barrier contemplates a structure which at least to some degree impedes release of a medicament, agent, etc. carried by the carrier, from the carrier. Selectively, as characteristics of the carrier warrant, a further functionality for the barrier per se, or the addition of a further element for the accessory article having the further functionality, depending upon the characteristics of the barrier, or barrier/carrier combination, is contemplated, namely, retention. As will later be developed, the barrier may be homogenous or heterogenous, e.g., monolithic or a composite, and is advantageously, but not necessarily, plastic/semi-plastic in nature.

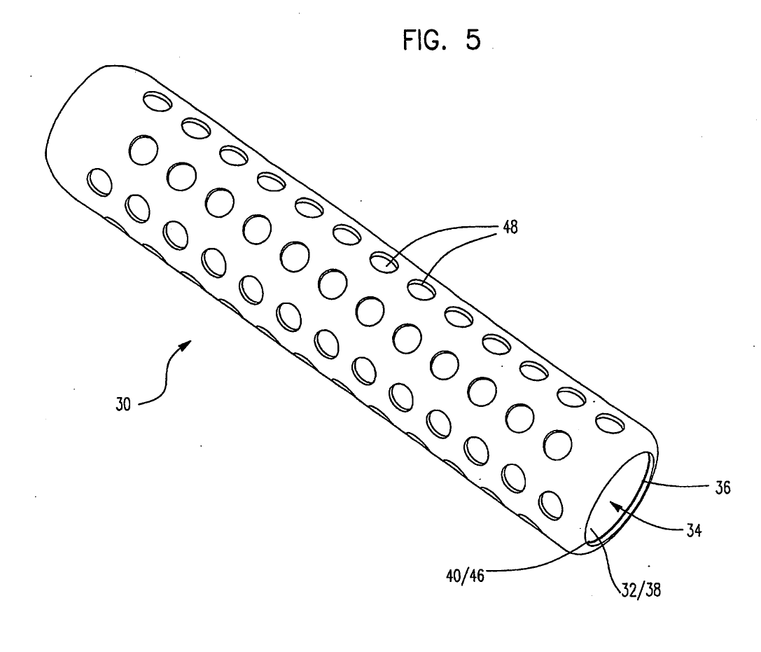

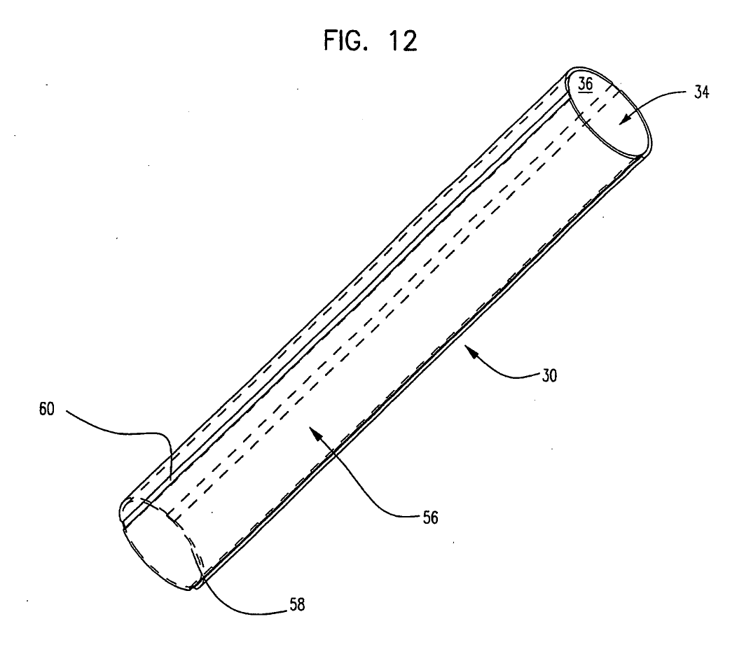

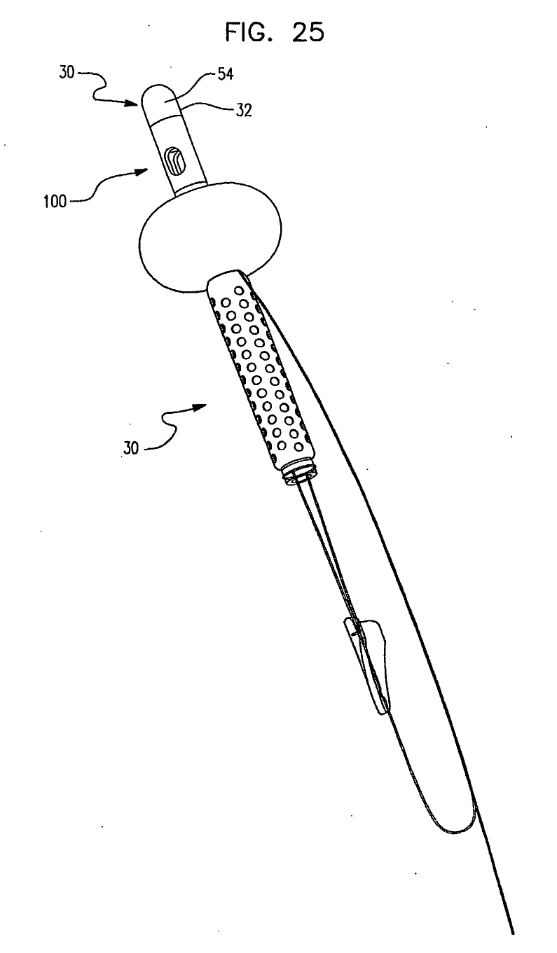

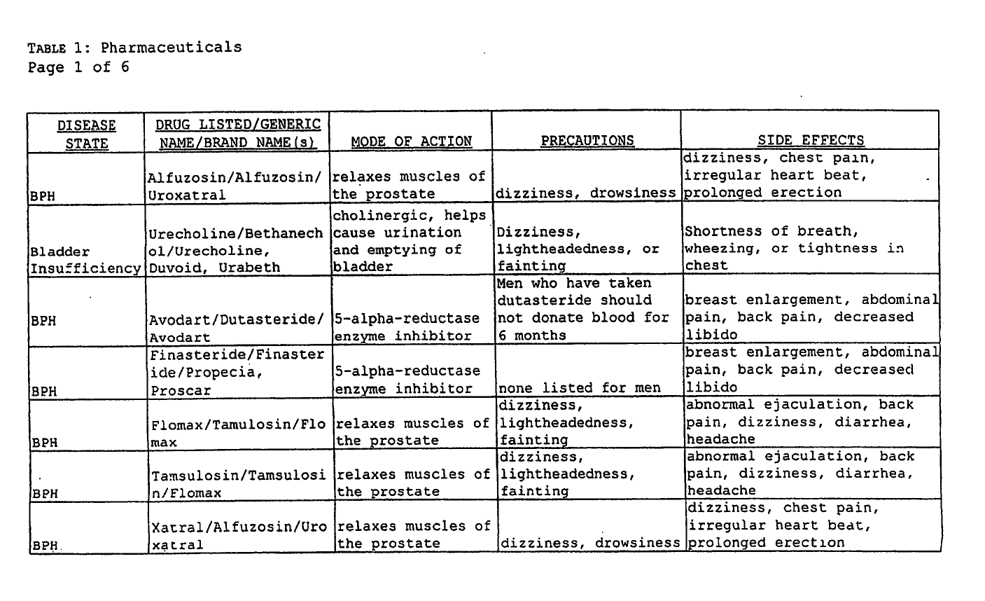

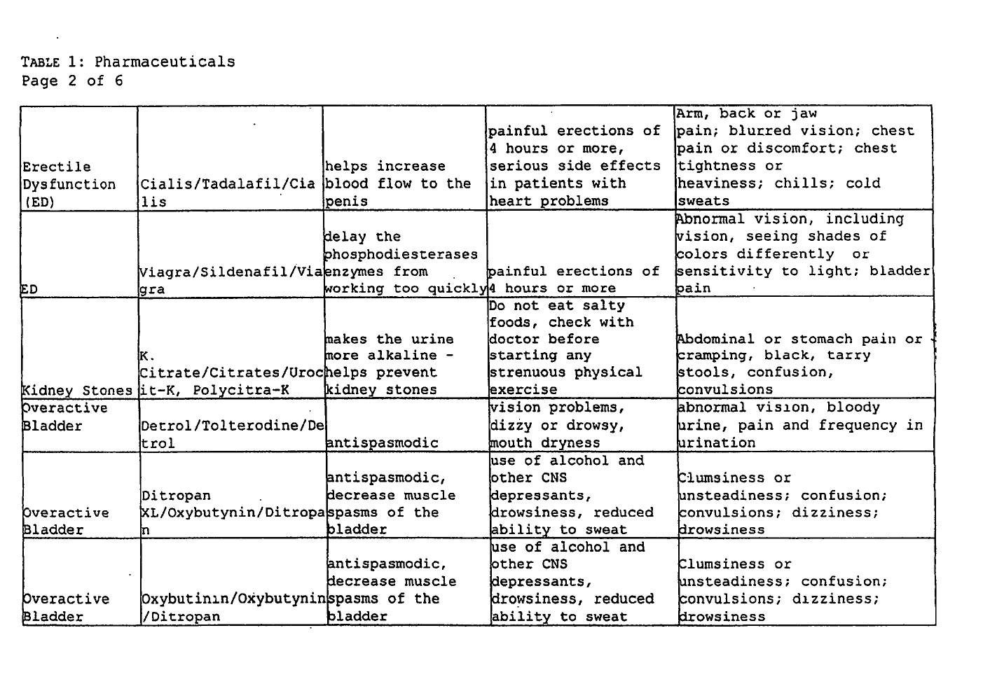

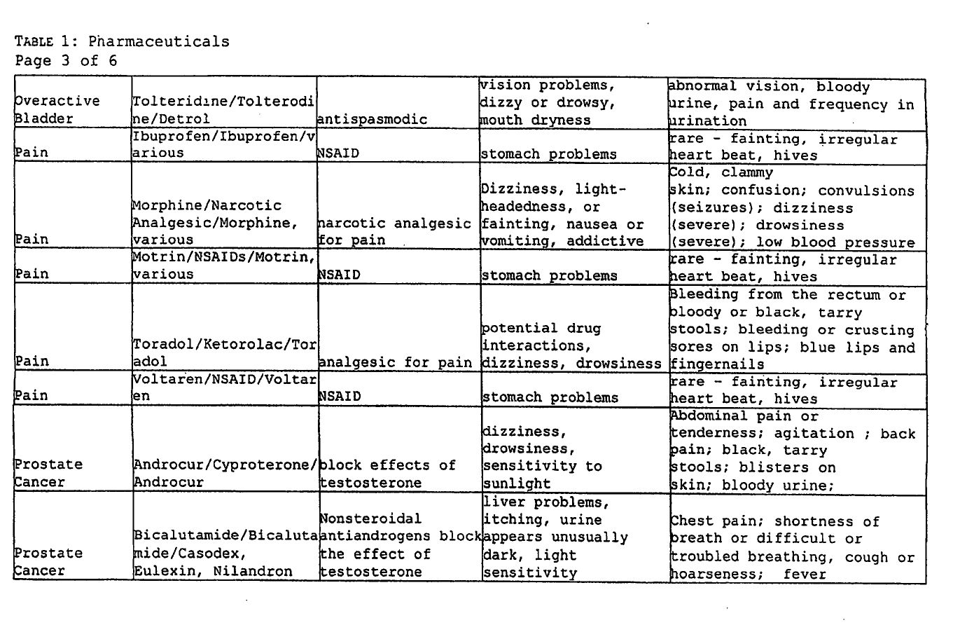

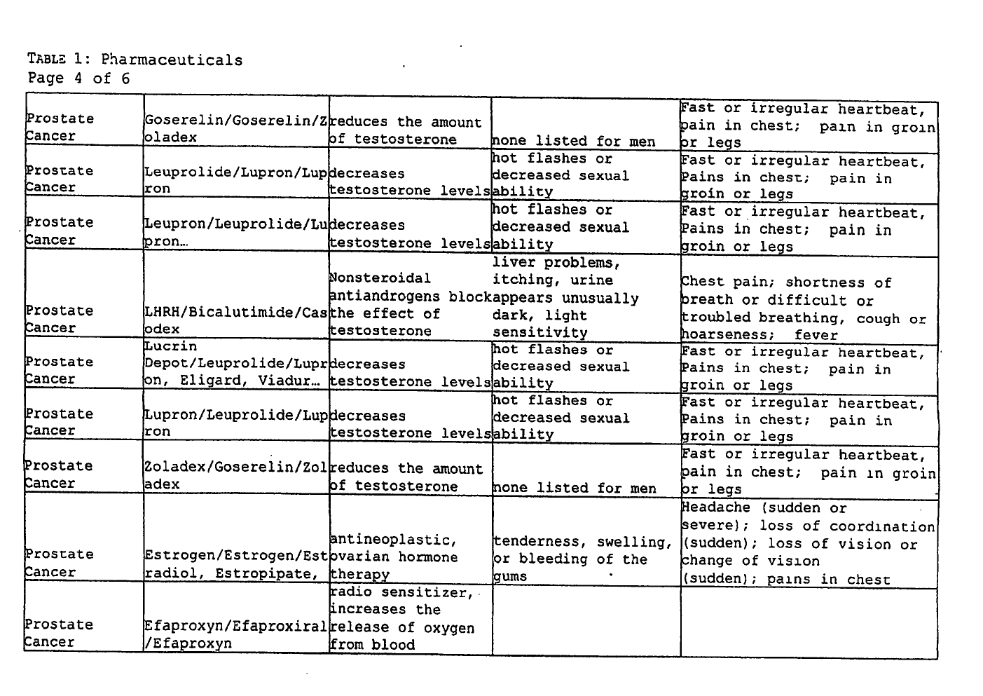

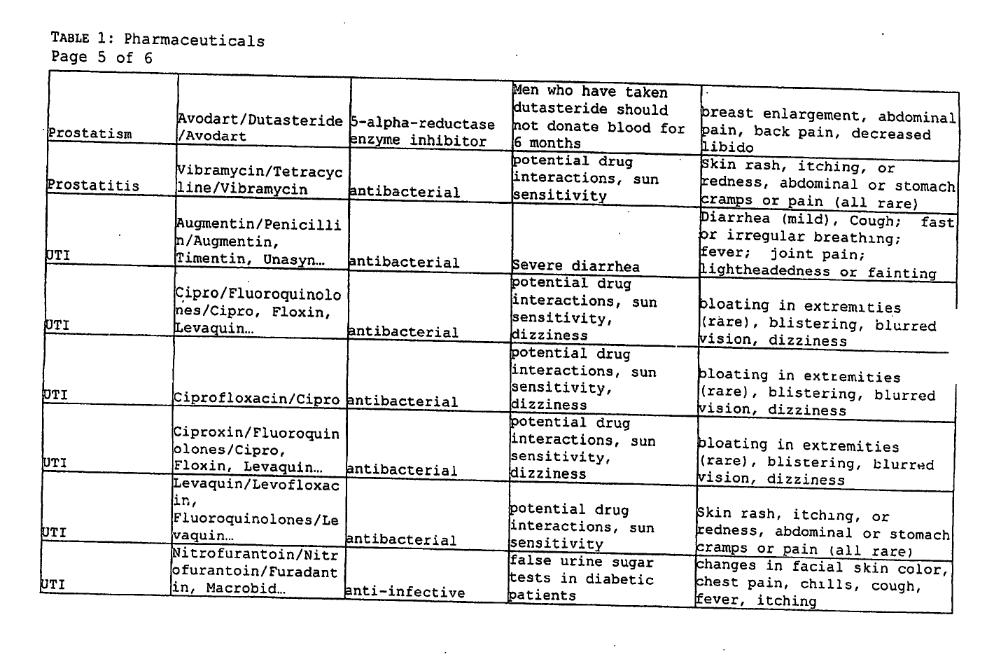

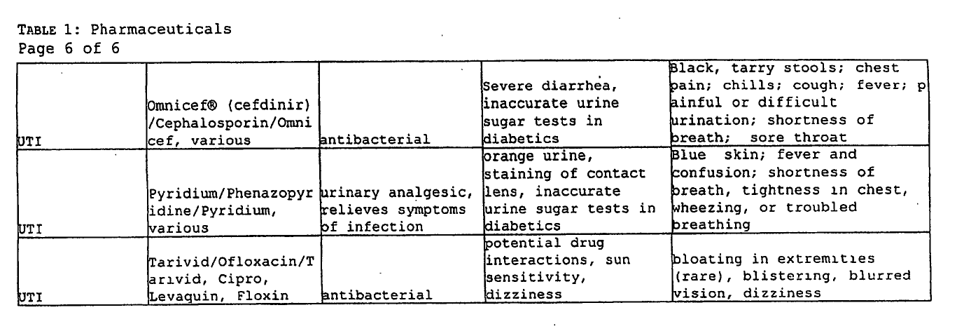

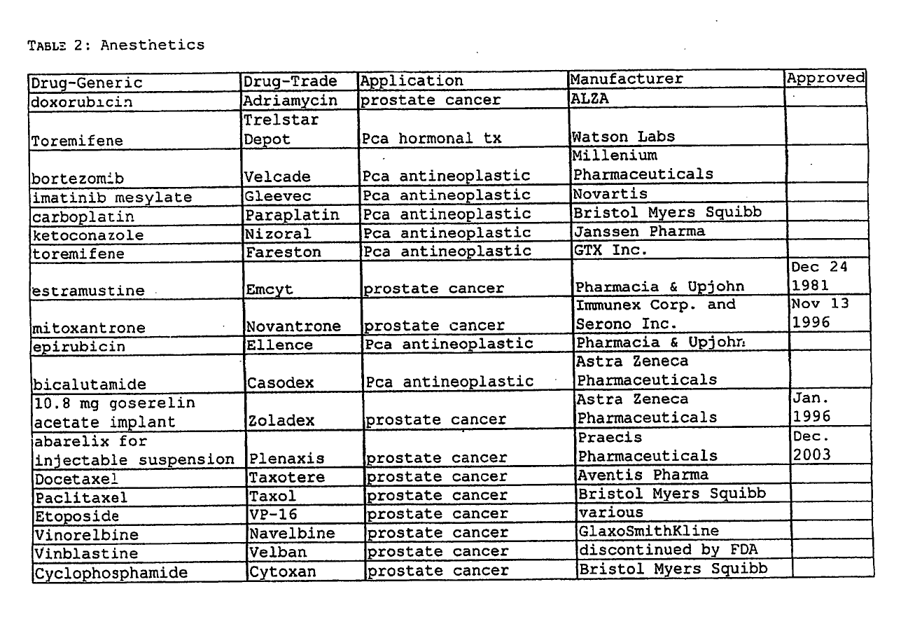

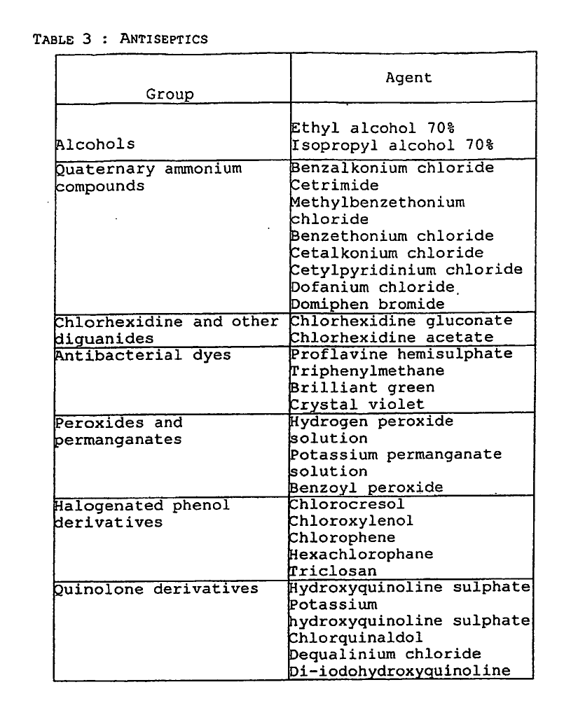

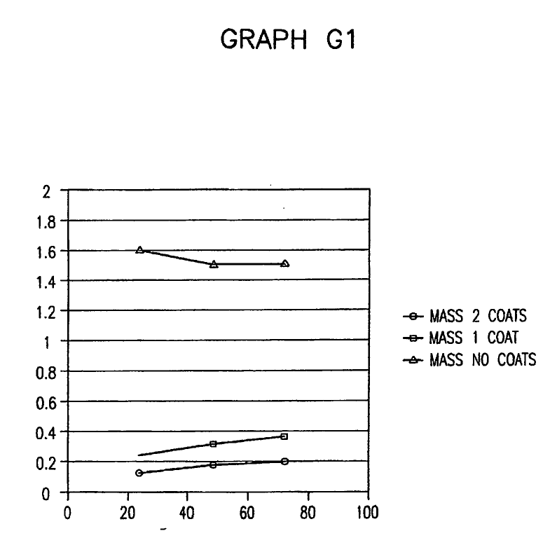

As previously noted, FIGS. 1-8, 12, and 25 are generally directed to accessory articles of the subject device. Notionally, the subject articles are characterized by a carrier, eluting element or reservoir, either alone, e.g., the articles of FIGS. 1-4, 12/13, and 25, or in combination with a barrier, e.g., the articles of FIGS. 5, 12/14, and 12/15. It is to be understood, as alluded to earlier, that as used throughout this description, in as much as “barrier” conjures up a mental image of an obstacle or impediment, it is more broadly meant to embrace the notion of a “covering” for the carrier (see e.g., the articles of FIG. 5, 9, or 12/15). As will be subsequently described, in the absence of an inherent retainment or affixation means for the carrier or article per se (see e.g., FIG. 25 and note the interference fit between the reservoir structure of FIG. 25 and the indwelling device), such means is advantageously provided in the form of the barrier (see e.g., the article of FIG. 9), or in lieu thereof, via a dedicated “retaining” element.

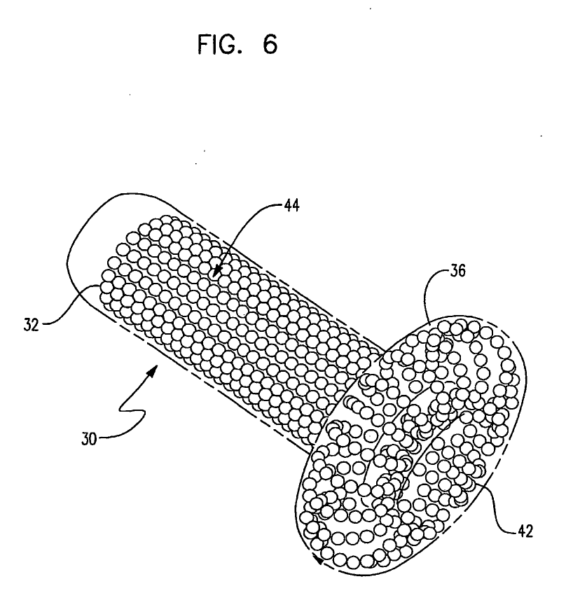

With particular reference to FIGS. 1-8, 12, and 25, the articles 30 in their fundamental form comprise a carrier 32, more particularly, a polymeric, active agent carrier, and an active agent carried thereby. The subject articles are preferably, but not necessarily, configured as tubular elements, e.g., an annular element such as sleeve or cylinder as shown in FIGS. 1-5, and 12, a capsule as shown in FIG. 25, a ring, collar, etc., or configurable as tubular elements (see the article configurations of FIGS. 6-8) as by “unrolling” the article from a “rolled-up” condition, such articles being characterized as torus shaped, i.e., as having a bagel or ring-like configuration. As should be readily appreciated, the configuration or geometry of the article is greatly influenced by a variety of factors or considerations, among other things, the inherent nature of the active agent, its relationship or “status” in connection with the carrier, and the nature of the indwelling device to which or upon which the article is to be affixed or supported.

With reference now to the articles of FIGS. 1-8 and 12, the carrier 32 includes a lumen 34 for receipt of at as least a portion of an indwelling medical device, more particularly, the carrier 32 includes an interior surface 36 for receipt upon an exterior surface of at least a portion of an indwelling device. The carrier 32 may be of homogeneous cross section, e.g., FIG. 1A, appearing as a lamina, or of heterogeneous cross section, e.g., FIG. 1A′, appearing as a composite or other fabrication which includes at least two lamina, e.g., a substrate 38 and covering or coating 40. It is to be further noted that, as the article of FIG. 24, the contemplated carrier or carriers of the subject invention may include, selectively or otherwise, voids, i.e. one or more bounded volumes, for the retention of an active agent. Such configuration has particular utility in cases where the agent is a liquid, or where the agent benefits from synergistic effects from exposure, combination, etc. with a liquid, for example, in a solution/solute approach to medicament delivery. Such articles feature solution storage and release capabilities, whether in the form of discrete or combined elements, structures, or subassemblies for the article.

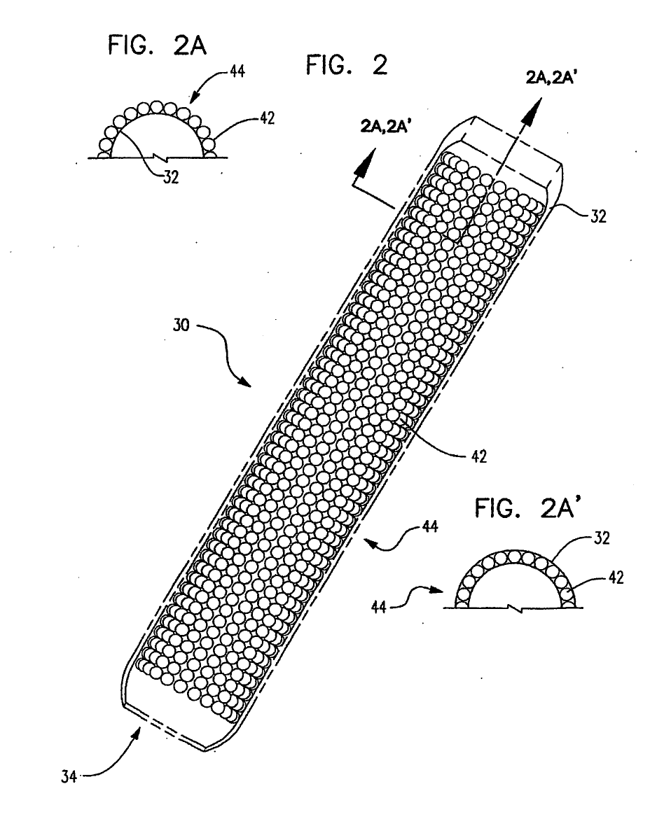

The carrier, or elements thereof as the case may be, are advantageously comprised of urethanes, more particularly, aliphatic, polycarbonate based thermoplastic polyurethanes, hydrophilic, aliphatic, polyether-based thermoplastic polyurethanes, or advantageously, combinations thereof; however, it is to be understood that carrier composition may be readily adapted to accommodate more or less lipophilic agents as a given therapy warrants. It is further, optionally contemplated that inorganic phases or pre-existing phases such as micro/macrospheres 42 (see e.g., the articles of FIG. 2 or 6), within the range of about 10-200 microns, be provided integral to the carrier, i.e., carrier structure, for example, as by encapsulation between and/or among select layers of the carrier (see FIG. 2A′, or 6). Similarly, a matrix of beads 44 may form a “covering,” i.e., a surface for the carrier. Such surface of covering may be a complete or partial, and may be intended to selectively cover a portion of an indwelling device.

In connection with FIG. 9, the article might advantageously include a covering, e.g., retainer 46, more particularly, a covering including perforations 48 or the like in furtherance of delivering a regulated supply of medicament from the carrier to the indwelling environment. It is further noted that the porosity of the subject structure may be selectively controlled via the inclusion of phase separating solutions, preferably those characterized by swift evaporation, or via adapted coverings or exterior lamina for the article. Advantageously, but not necessarily, the carrier, and perhaps the barrier, is microporous, with open pores on its exterior and perhaps at least part way through same. The size of the pores, the viscosity of the agent, the physical relationship between the agent and the walls of the pores (e.g., mutually attractive, neutrally attractive, or repulsive, such as based upon their relative hydrophilicity or hydrophobicity) will assist in determining or tailoring the rate of release of the agent into the biological system into which the drug delivery element is introduced.

As to structure fabrication, the layers thereof may be applied by dipping, spraying or layering pre-made sheets into tubular structures. These layers may be composed of any of solvent castable polymeric materials including, polyvinylclorides, polyethlyacrylates, polyvinylnitriles and preferably polyurethanes as well as curable materials as water based latexes.

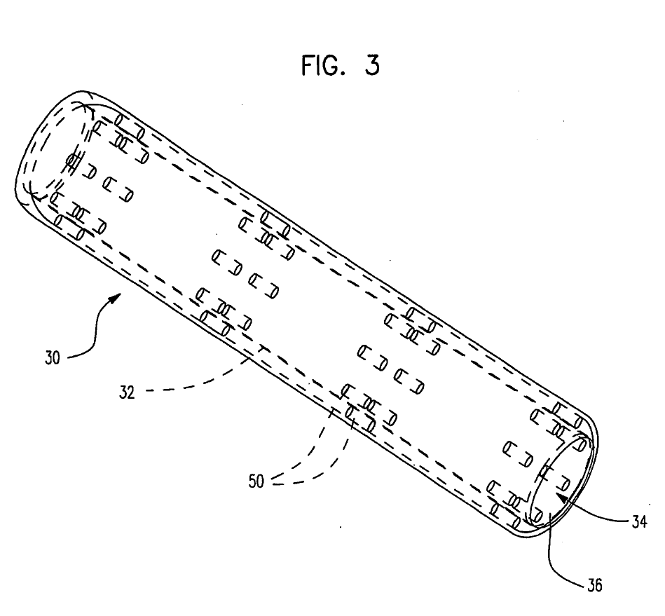

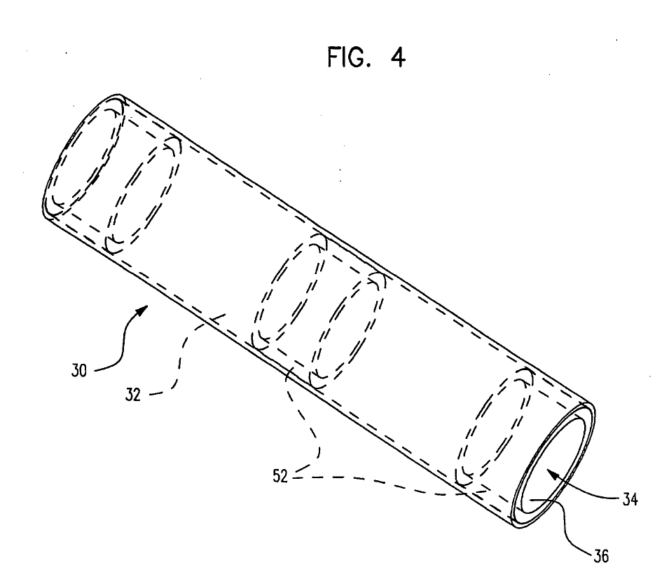

Referring now particularly to FIGS. 3 & 4, accessory articles or modular delivery systems 30 are illustrated for energy delivery, i.e., the physiologically active agent is in the form of energy emitted by radioactive elements. As with the heretofore described articles, the modular delivery system, more particularly the carrier 32 thereof, is preferably configured as an annular element with radiation therapy seeds 50 supported thereby or therein (FIG. 3), or equipped with bands or collars 52 (FIG. 4), more particularly, but not necessarily, brachytherapy bands, e.g., bands carrying iodine 125 or the like. It is to be noted that the article of FIG. 4 need not have utility only in connection to energy delivery, that is to say, it is likewise contemplated that the bands or collars, or equivalents thereof, function as a carrier in a general sense.

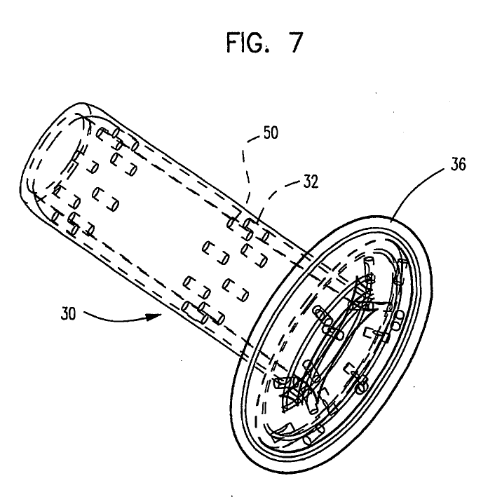

In connection to FIG. 3, while seeds 50 are generally shown as being cylindrical in shape, they may be nearly any shape including, but not limited to rectangular or spherical, in furtherance of delivering an effective quantity, intensity and pattern of and radiation. In connection to FIG. 4, the “seeds,” or functional equivalents thereof are generally depicted as spaced apart cylindrical sleeves 52 upon a carrier 32, more particularly a substrate. This configuration provides the advantage of a greater single mass concentration, however, a complete cylindrical section does not provide for a rolled up configuration and attendant rolled-out deployment (but see FIG. 7). As should be appreciated, the bands may be specifically positioned in or throughout the article in order to provide the agent, e.g., solution, energy, etc., directly to a target region. Conversely, critical, sensitive or otherwise select areas may be avoided via appropriate positioning and/or spacing.

As an example of the utility, it may be beneficial to introduce solution or energy to the center portion of the urethra in the region surrounded by the prostate, or near the median lobe of the prostate that is near the bladder, while avoiding the external sphincter. Unless the external sphincter is infected or behaving in a non-synchronous manner it is always desirable to avoid it in procedures because it controls continence.

Contemplated carriers of the subject invention may be inherently affixable upon the portion of the indwelling device, adapted for affixation upon the portion of the indwelling device, or affixable upon the portion of the indwelling device via a retainer (see, e.g., the article of FIG. 9, and also that of FIG. 5 wherein the “retainer” is further functioning as barrier or facilitating layer. For example, the article of, for example, FIG. 1 may have a characteristic radial resiliency, or other characteristic and/or property, such that the article is tensioningly received upon a portion of the indwelling device. Equally suitable, the inner surface of the article, i.e., that delimiting a device receiving lumen, may include adhesion means (e.g., chemical or mechanical), or be generally adapted to cooperatively engage a portion of the indwelling device. Finally, as noted in connection to FIG. 9, a retainer may suitably cover at least some portion of the carrier and hold in place upon the indwelling device.

With reference now to FIGS. 24 & 25, the article 30 depicted is characterized as a capsule, more particularly, a fluid filled or fillable bulb. The carrier 32 of the article includes a reservoir 54, more particularly, a void or fixed volume. The fluid may either be pre-filled, or filled using a suitable small gauge needle and syringe. The bulb is generally adapted for passive/active release of fluid as circumstances warrant, and is likewise adapted such that an indwelling device may be quickly and securely equipped with same, as for instance via receipt and retention of a profiled free end of the bulb. The configuration as illustrated, as well as equivalents thereto, whether mechanical or chemical in nature, impart the sought after modular functionality for the article.

As is appreciated with reference to FIG. 25, the article of FIG. 24 provides for the delivery of fluid into the bladder with minimal contact with the bladder surface. The endourethral device 100 may also be readily adapted to otherwise support the article, or variants thereof, with the device equipped with a combination of articles, i.e., a bulb and sleeve, as shown.

As to the voids and/or reservoirs generally, the bulb may be constructed of one or more internal cavities to allow for sequential or synchronous release of one or more agents, e.g., solutions, with agent release rate controlled or regulated by the carrier/barrier properties of the article. As should be readily appreciated, the bulb may be adapted or otherwise constructed so as to include a passage suitable to connect and thereby communicate with fluid storage or release elements provided elsewhere on the device, whether they're in the form of a modular article as heretofore described, or integral to the device. For example, fluid for this proximal bulb may communicate with the surfaces and sleeves heretofore described. Fluid may be passively moved in any direction based on the pressure and concentration motivations.

Heretofore described articles of the subject invention are noted as possessing an elasticity or resiliency, either imparted by the properties of the carrier, or other lamina associated with the article. The subject articles leverage the ability of polymers with a high strain limit and elongation limit (i.e. a silicone rubber) to reversibly extend or stretch, radially and/or axially, and then return to their original shape as means to be delivered, located, and then mounted on an indwelling device, e.g., a catheter, endourtheral device, etc.

The articles of FIGS. 12-15 depart from those previously described in that they are characterized as possessing controlled strain properties, features or functionality. The subsequently described articles utilize a combination of elements or features such that the deformation for application of the article to the device is controlled and/or limited. As might be appreciated, notionally, the carrier, when functioning as a substrate, is to be minimally disruptive to any lamina overlaying same, i.e., lamina overlaying the substrate are to be minimally impacted by an altered condition of the substrate.

With reference to FIG. 12, a controlled strain elastic sleeve configuration is generally shown for a homogenous element 30. The sleeve has three primary functional areas, namely, a limited strain area or section 56, a coating or layer for agent delivery 58, a high strain section 60. Alternate sectional views for this contemplated article are provided in FIGS. 13-15. While the preferential strain sections 60 are delimited as longitudinally extending grooves or slots, such zone or zones need not be so limited in practice.

In connection to FIG. 13, it can be seen that the two strain sections 56, 60 vary in circumferential cross section, a necessity for a homogeneous sleeve. The variance in cross section directs the strain in the sleeve to occur primarily in the high strain areas (i.e., 60) when the sleeve is expanded in preparation for delivery on to a device. The amount of strain occurring in the wall is proportional to the hoop stress in the wall and similarly the amount of hoop stress is proportional to the wall cross section at the specific location, circumferentially, in the sleeve. Selectively reducing the wall thickness in specific areas provides a means to control the location of strain in the tube during radial expansion.

As previously alluded to, controlling the amount of strain in the sleeve is important due to the fact that some compound delivery coatings are polymers with a low strain limit. This strain limit may be below the amount of strain required to expand the sleeve for delivery and location on the intended device. If the coating under goes too much strain during delivery, the compound release properties of the coating may be altered, or disabled altogether. Managing the strain on the sleeve to specific areas allows the sleeve to be expanded and applied upon a device while maintaining, or limiting an altered state for the delivery mechanism of the article, i.e., the overall integrity thereof.

An alternate, non-limiting fabrication for the article of FIG. 12 is shown in FIG. 14. The hybrid sleeve 30 includes a high strain section formed by a high strain inner member 62 and the limited strain section formed by the agent carrying member 58 attached to the inner member. This construction allows for a variety of, compound delivery mechanism to be used with a common inner member.

A further alternate, non-limiting fabrication for the article of FIG. 12 is shown in FIG. 15. A sleeve assembly 30 is generally depicted wherein the agent carrying member 58 is contained within an annular gap 64 of inner and outer elements 66, 68. In this configuration, the inner and outer elements are responsive to strain imparted upon the article while the agent carrying member remains interposed therebetween, and substantially unaltered. As shown, there can be greater than one carrier, e.g., a bead type matrix (FIG. 9), each carrier providing different compound delivery capabilities. The outer member may or may not participate in the release mechanism of the carrier.

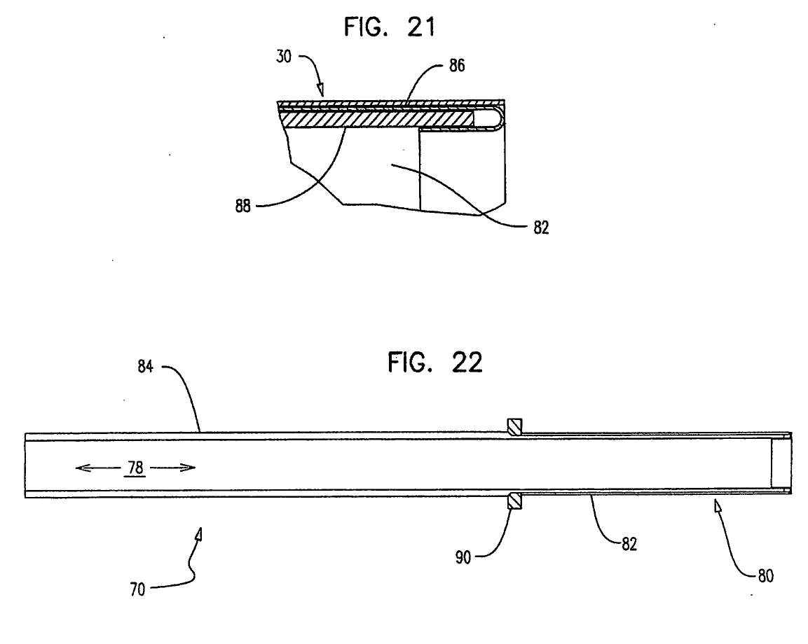

With regard to the article of FIG. 12, or more generally, any of the heretofore described articles which do no lend to a being configurable as a tubular element (i.e., a rolled article, see e.g., the articles of any of FIGS. 6-8), i.e., those that are configured as a tubular element, more particularly, a sleeve, a variety of delivery mechanisms and attendant applicators are contemplated. Three device application mechanisms or approaches are subsequently described, namely, coil applicator (FIGS. 16-19), inverted or retractable applicator (FIGS. 21 & 23), and “slip-fit” applicator.

The subject delivery systems generally provide an applicator having a article receiving portion, i.e., a mandrel for retaining and maintaining the received article in a pre-applied state or condition, namely, radially expanded. The applicator, or mandrel is generally configured or otherwise adapted to receive at least a portion of the device, e.g., the applicator or mandrel itself is sleeve-like, i.e., is characterized as having a device receiving lumen. With such application assembly, the article may be transferred from the applicator or mandrel, to the underlying device.

The interface between the article and applicator is suitably one of interference, adhesion, friction, tension, or combinations of same. As should be appreciated, the particulars of the interface are a function of a variety of considerations, with material properties generally being primary or controlling.

With general reference to FIGS. 16-19, a sleeve structured medicament delivery article 30 is shown supported upon and by a portion of an applicator 70, more particularly, a wound element, e.g., a coil, so as to form an assembly or system 72. Advantageously, but not necessarily, the wound element may be reinforced or selectively strengthened in known ways, e.g, via a the inclusion of a membrane, application of a coating, etc., so as to impart sufficient structural integrity, either radially or axially, to/for the applicator, and thus the assembly. Furthermore, the configuration or pattern of the wound element may be modified, based on the sought after removal characteristics, for example, the wound element may be configured as a split coil with a deployment handle on each end or winding.

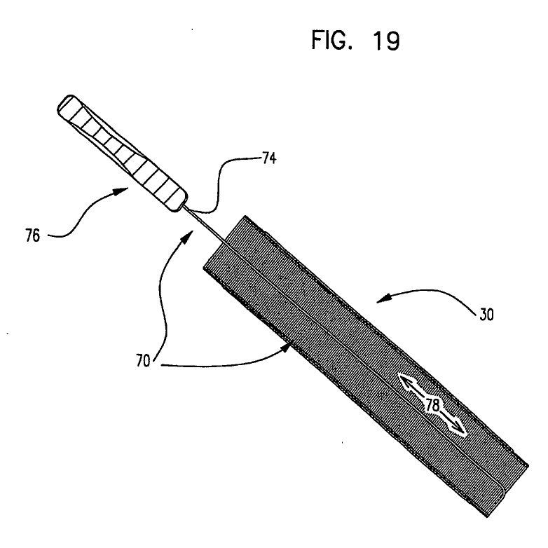

An end of the opposing ends of the coil, i.e., a free end 74, extends from the sleeve-like structure, and terminates in a handle or grip 76 (FIG. 16 or 19). As will be appreciated in connection to a description of the application mechanism of the subject assembly, the free end of the coil may extend in a direction away from the windings of the coil (FIG. 16), or may extend toward the other end of the coil, and beyond the windings thereof, for example through the lumen 34 of the article 30 (FIG. 19).

Positioning the assembly 72, relative to a device 100, for applying or otherwise delivering the article 30 to or upon the device is generally shown in FIG. 17, more particularly, the supported article is illustrated in a position to overlay a stent body of an endourethral device. While the proximal end of the device is preferably received into a lumen 78 of the applicator 70 opposite the handle 76, it need not be limited to such insertion, the arrangement as shown between the assembly and the device being the primary focus of the exercise.

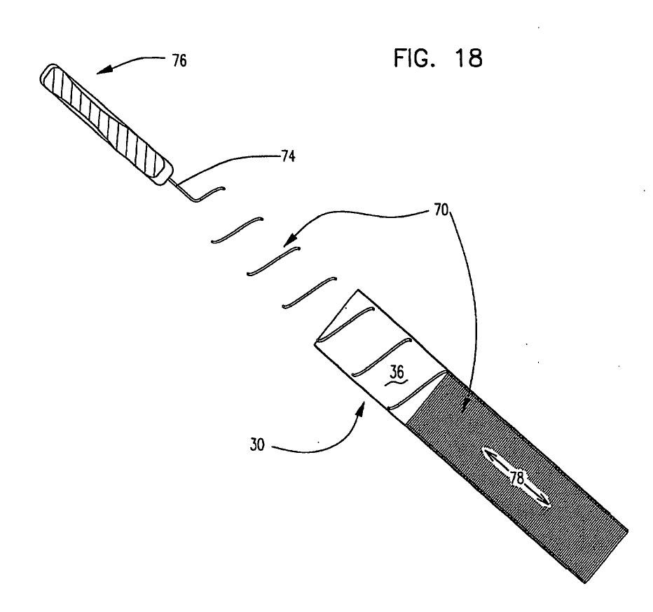

With the sought after positioning of the assembly relative to the device, tension may be applied to the handle 76 of the applicator 70 as indicated in FIG. 17 so as to unwind the windings of the wound element as generally shown in FIG. 18. As the coil unwinds, it is removed from beneath the medicament delivery article 30, with the article thereby sequentially transferring from the applicator 70 to the device 100. In connection to the assembly of FIG. 16, the union of the article upon the device proceeds in a proximal to distal direction relative to the handle, whereas an opposite union between the structures, i.e., distal to proximal, is imparted via the assembly of FIG. 19.

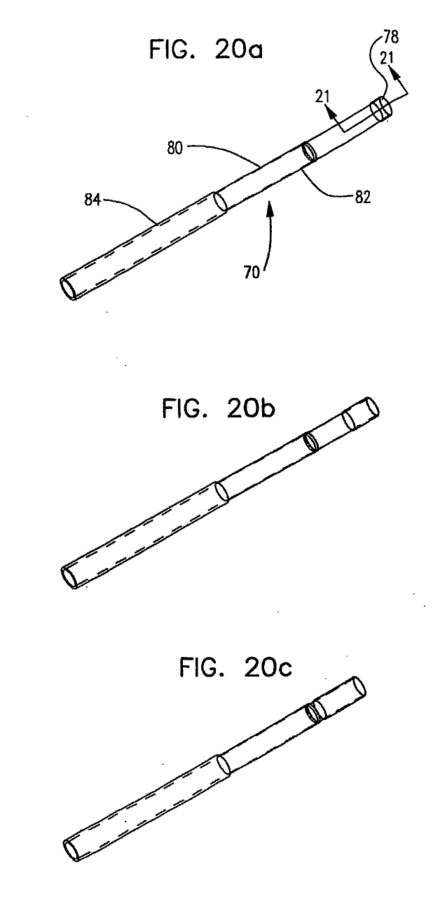

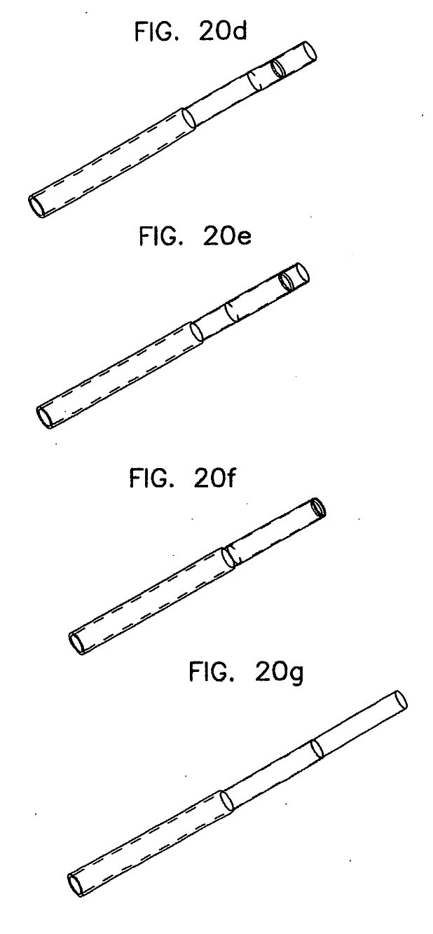

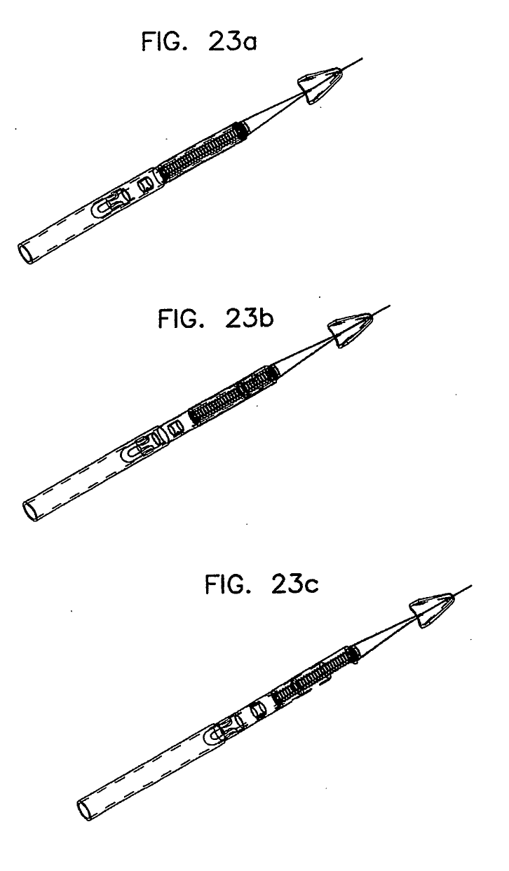

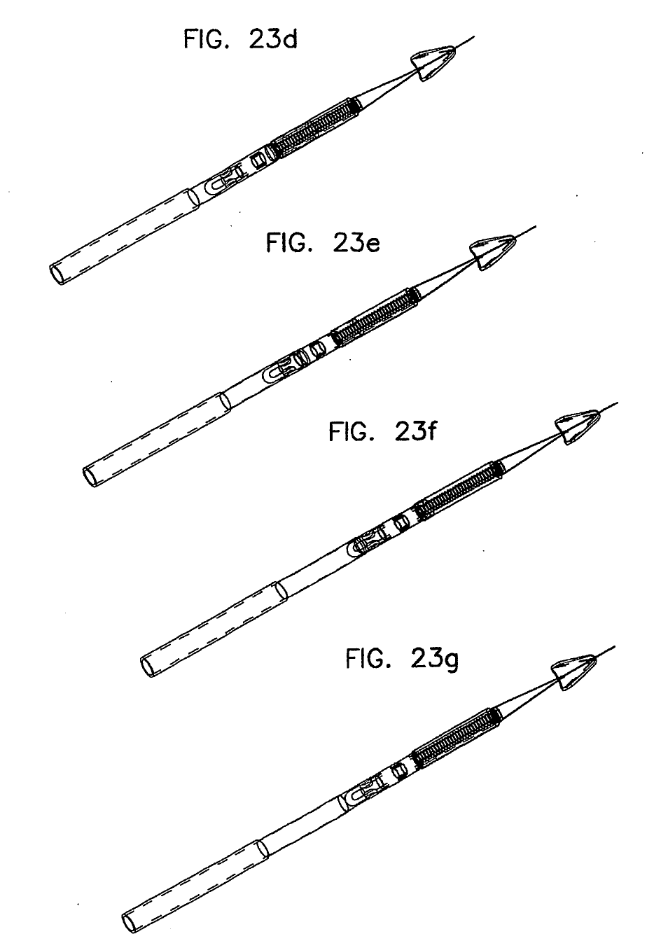

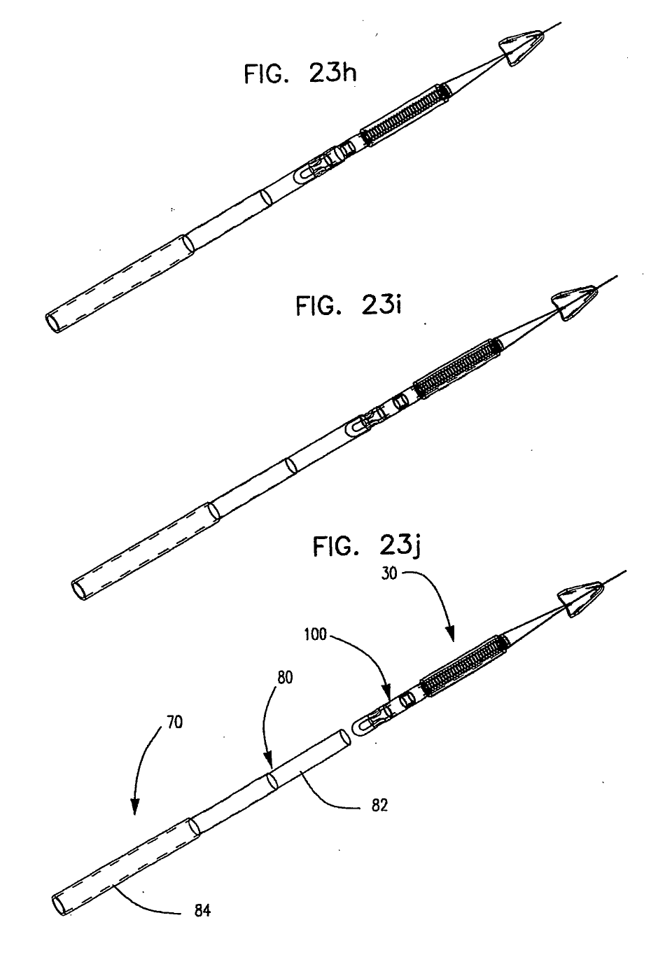

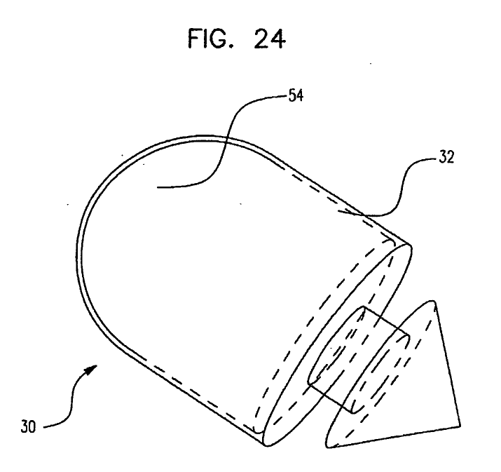

With general reference now to FIGS. 20-23, an applicator 70 is shown in FIG. 20 in a variety of functional states (a)-(g), an article receiving end or mandrel 80 thereof supporting a medicament delivery article 30, and the contemplated assembly shown in a variety of functional states (a)-(j) in FIG. 23, generally correlating to the states (a)-(g) of FIG. 20, relating to applying the article to the device depicted.

The subject applicator 70 is generally characterized as having invertible, i.e., retractable or rolling, and rigid portions 82, 84. As will be subsequently discussed, the subject invertible portion 82 is transformable from a fully-retracted condition upon the rigid portion 84, to a fully extended condition beyond the extent of the rigid portion. The subject roll back feature enables axial sliding of the sleeve portions relative to each other, and segments of the article receiving portion relative to each other.

With particular reference to the applicator of FIG. 20, the applicator 70 is generally configured as an annular element or sleeve having first and second portions 82, 84 (FIG. 20(g)). Generally, the portions characteristically have differing degrees of rigidity, particularly, radial rigidity. As best seen in connection to FIG. 20(a), the relatively less rigid portion 82 is selectively, reversibly and progressively supportable upon a segment of the relatively more rigid portion 84 of the sleeve, with the configurations of FIGS. 20(b)-(f) depicting various states of extension for the article transferring sleeve portion 80 relative to the other article portions 82, 84.

Notionally, a medicament delivery article is received upon the applicator configuration of FIG. 20(a), with a portion of an indwelling device received within the lumen 78 of the applicator 70 of the contemplated assembly 72 so configured (see FIG. 23(a)), wherein the entirety of the relatively less rigid portion 82 is rolled back upon the more rigid portion 84, both the article 30 and the article receiving portion 80 being thereby fully supported. As should be readily appreciated in connection to FIGS. 23(a)-(g), the assembly is manipulated such that the article and sleeve portion underlying same, is progressively rolled-off and out from the relatively more rigid sleeve portion (FIGS. 23(b)-(d)), and preferably, but not necessarily, further progressively rolled-off from the doubled back less rigid sleeve portion of the applicator (FIGS. 23(e)-(g), where after the indwelling device, now equipped with the medicament article, is removed from the lumen of the applicator (FIG. 23(i)-(j)).

In addition to the construct of FIG. 20, an alternate arrangement for the article receiving end portion or mandrel 80 of the sleeve is shown in an alternate partial section of FIG. 20(a), namely, FIG. 21. The article receiving and transferring sleeve portion is indicated as having distal 86 and proximal 88 portions which delimit a slideable interface therebetween.

In connection with equipping an indwelling device with a medicament delivery article utilizing the applicator of FIG. 20, a push ring or collar 90 is further, and optionally contemplated (see FIG. 22, an alternate version of the applicator of FIG. 20(g)) in furtherance of facilitating delivery of article of the assembly. The ring, or equivalent structure, provides a mechanism whereby, rather “pulling” a proximal portion of the sleeve while the article and sleeve receiving portion underlaying same is static, the article may be “pushed” from the free end of the applicator and onto the device.

Finally, in connection to a “slip-fit” applicator, an article receiving portion of the applicator, i.e., a free end or mandrel portion thereof as generally configured as that shown in FIG. 20(g), is generally adapted to provide a slip plane or low friction cooperative engagement between that portion of the sleeve and the article to be transferred to the device. As previously discussed, a push ring may be used to facilitate delivery of the article. The article receiving portion of the sheath preferably uses a low friction material or coating to facilitate axial sliding of the article from the free end of the applicator, to the device. It should be appreciated that such adaptations nonetheless require a non-reversible engagement of the article upon the device.

In connection with utilization of the subject medicament delivery article/system with endourethral devices, testing was undertaken so as to determine urethral endothelium penetration ability of a variety of agents which share some or similar properties of agents of interest, namely, finasteride, mitoxantrone, and ciprofloxacin. As to materials, acidic, basic or lipophilic dyes, with molecular weights in the range of 3-400, were used, including: crystal violet (MW 408); basic Nile blue (MW 300); Nile red lipophilic; oil red O lipophilic; erythrosin (MW 890) acidic; and, methylene blue (MW 319) acidic. Mitoxantrone, and ciprofloxacin were likewise tested. The dyes and agents were mixed in a thermoplastic polyurethane (TPU) matrix comprising of 30/70 weight ratio of Carbothane® 72D, an aliphatic, polycarbonate based TPU, to Tecophil®, a hydrophilic, aliphatic, polyether-based TPU (Noveon, Inc.) in a 50/50 mixture of DMAC and THF. The polymers were prepared, 5% by weight, with dyes or agents added to the mixture as an additional 1% by weight.

Dyes and drugs were first coated onto silicone rubber sheets as droplets, and dried at 70° C. overnight. To gain a first approximation of the ability of the dyes to penetrate tissue, the sheets were placed against the outer membrane of calf liver, either for 24 or 48 hours. The tissue was placed on top of the sheet and supplied with nothing more that the pressure of the tissue against the sheet.

Human prostate tissue was obtain postmortem, and within 24 hours thereof, and flash froze. The tissue was thereafter thawed at ambient temperature, and used within 12 hours. The tissue was not fixed.

Six dyes, namely, methylene blue, crystal violet, a combination of Nile blue and Nile red, oil red O, and erythrosin were coated onto two stents in the form of 3 mm bands or rings. One stent was coated with both mitoxantrone, and ciprofloxacin. Thereafter, the stents were placed in a phosphate buffered Ringer's solution with 1% virocide into excised prostates for 24 and 48 hours at 37° C. Prostates were removed from containers and dissected axially along the urethra, and then at right angles transversally through center portion of the dyes.

In connection with the liver tissue of the approximation methodology, the basic dye crystal violet penetrated the tissue to the greatest extent, however, the acidic dyes also penetrated. Somewhat unexpectedly, the lipophilic dyes oil red O and Nile red only mildly stained the tissue, suggesting that penetration of polar dyes is not inhibited, whether slightly positive or negative, and that membrane structure may be inhibitory to lipophilic dyes.

In connection with the prostatic tissue, after 48 hours, crystal violet stained most prominently, with oil red O and Nile red staining the least. Nile blue, methylene blue and erythrosin were approximately equivalent. Penetration of the dyes at 24 hours was slightly less than that exhibited at 48 hours.

In connection with the medicaments, i.e., mitoxantrone and ciprofloxacin, the delivery system comprised a mixture of 20% by weight of either of the drugs, in 72D Carbothane Tecophilic (Noveon, Inc.). The polymers where prepared as a 5% by weight in THF and DMAC. Each of the mixtures were applied in a spaced depart condition upon a prostatic stent so as to coat 1 cm segments thereof. Thereafter, the stent was placed in a excised prostate for 48 hours. The prostate with the inserted stent was bathed in 50 ml Ringer's solution, with 0.2% virocide to inhibit bacterial growth. The prostate was removed from the test solution, and, with the stent removed, axially bisected. Prosthetic tissue specimens were further prepared of section cut perpendicular to the urethra and thereafter viewed under florescent light.

Tissue staining was noted particularly around glandular membranes. Both drugs were generally detected throughout the section, with a greater concentration on surfaces open to glands, ducts, or capillaries. It is not known if the higher concentration was a result of diffusion to these areas or by conduction through open regions. In either case, the drugs were, more or less, uniformly present. It is not known how far the drug diffused laterally away from the edges of the coated stent sections, nor the degree of interference of one drug with another. As the coated sections of the device were approximately 1-1.5 cm apart, and the appearance and location of the drugs appeared somewhat different in each section, it is believed that each section contains predominantly one drug. This is consistent with the result of dye staining which visually showed that various dyes penetrate to a depth of several millimeters, and utilizing microscopy, to depths of at least 10 mm.

Ongoing work contemplates coatings with a higher medicament loading, e.g., approximately 10 mg of ciprofloxacin, in combination with a 72D Carbothane barrier coating of approximately 15-30 microns in thickness, as well as a 95A Carbothane barrier coating. A 60D Tecophilic matrix is further contemplated.

In connection with elution rates, agent release as a function of time is illustrated in Graph G1. Elution rates for a polymeric coating containing 1.6 mg of ciprofloxacin is shown, more particularly, data points associated with: the polymeric coating with alone; the polymeric coating with a single barrier layer of 72D Carbothane; or, the polymeric coating with a two barrier layers of 72D Carbothane. The data points are associated with release at 24, 48 and 72 hours. The subject outcomes, and those implicated therefrom, verify that solution introduction adjacent prostatic tissue will result in the uptake of the select solution into the prostatic gland. This is critical for efficacy.

There are other variations of this invention which will become obvious to those skilled in the art. It will be understood that this disclosure, in many respects, is only illustrative. Although the various aspects of the present invention have been described with respect to various preferred embodiments thereof, it will be understood that the invention is entitled to protection within the full scope of the appended claims.

What is claimed is:

1. An adjunctive accessory article for controlled release of at least a single physiologically active agent from an indwelling medical device so equipped, said article comprising a polymeric active agent carrier and an active agent carried thereby, said article configurable as a tubular element upon a portion of the indwelling medical device.

2. The adjunctive accessory article of claim 1 wherein said polymeric active agent carrier comprises a laminate.

3. The adjunctive accessory article of claim 1 wherein said polymeric active agent carrier comprises a matrix.

4. The adjunctive accessory article of claim 1 wherein said polymeric active agent carrier comprises a composite.

5. The adjunctive accessory article of claim 1 wherein said polymeric active agent carrier comprises an aliphatic, polycarbonate based thermoplastic polyurethane.

6. The adjunctive accessory article of claim 5 wherein said polymeric active agent carrier comprises a hydrophilic, aliphatic, polyether-based thermoplastic polyurethane.

7. The adjunctive accessory article of claim 1 wherein said polymeric active agent carrier comprises a hydrophilic, aliphatic, polyether-based thermoplastic polyurethane.

8. The adjunctive accessory article of claim 1 wherein said polymeric active agent carrier is inherently affixable upon the portion of the indwelling device.

9. The adjunctive accessory article of claim 1 wherein said polymeric active agent carrier is adapted for affixation upon the portion of the indwelling device.

10. The adjunctive accessory article of claim 1 wherein said polymeric active agent carrier affixable upon the portion of the indwelling device via a retainer.

11. The adjunctive accessory article of claim 10 wherein said retainer comprises an annular element.

12. The adjunctive accessory article of claim 10 wherein said retainer is adapted to regulate elution of said active agent from said active agent carrier.

13. The adjunctive accessory article of claim 12 wherein said retainer comprises an annular element.

14. The adjunctive accessory article of claim 12 wherein said annular element is resilient.

15. The adjunctive accessory article of claim 1 wherein said polymeric active agent carrier comprises a substrate.

16. The adjunctive accessory article of claim 15 further comprising an active agent barrier overlying said substrate for controlled prolonged release of said active agent from said substrate.

17. The adjunctive accessory article of claim 16 wherein said substrate comprises a lamina.

18. The adjunctive accessory article of claim 16 wherein said substrate comprises a composite.

19. The adjunctive accessory article of claim i6 wherein said substrate comprises a matrix.

20. The adjunctive accessory article of claim 1 further comprising an active agent barrier overlying said polymeric active agent carrier to facilitate controlled prolonged release of said active agent from said polymeric active agent carrier.

21. The adjunctive accessory article of claim 21 wherein said active agent barrier comprises a laminate.

22. The adjunctive accessory article of claim 21 wherein said active agent barrier comprises a coating.

23. The adjunctive accessory article of claim 22 herein said active agent barrier comprises a single coating.

24. The adjunctive accessory article of claim 22 wherein said active agent barrier comprises at least two coatings.

25. The adjunctive accessory article of claim 1 wherein said active agent comprises a solution.

26. The adjunctive accessory article of claim 1 wherein said active agent comprises an antibiotic.

27. The adjunctive accessory article of claim 1 wherein said active agent comprises an anti-inflammatory.

28. The adjunctive accessory article of claim 1 wherein said active agent comprises an antiseptic.

29. The adjunctive accessory article of claim 1 wherein said active agent comprises an analgesic.

30. The adjunctive accessory article of claim 1 wherein said active agent comprises an antineoplastic.

31. The adjunctive accessory article of claim 1 wherein said active agent comprises one or more pharmaceuticals.

32. The adjunctive accessory article of claim 1 in combination with an applicator, said article retained upon a portion of said applicator in furtherance of equipping the indwelling device with same.

33. The adjunctive accessory article of claim 32 wherein said applicator comprises a article receiving segment.

34. The adjunctive accessory article of claim 33 wherein said article receiving segment of said applicator comprises a wound element.

35. The adjunctive accessory article of claim 34 wherein said wound element is selectively unwound in furtherance of article transference from said applicator to the indwelling device.

36. The adjunctive accessory article of claim 33 wherein said article receiving segment of said applicator comprises an invertible end portion thereof.

37. The adjunctive accessory article of claim 33 wherein said article receiving segment of said applicator includes a surface adapted for sliding transfer of article supportable thereby.

38. The adjunctive accessory article of claim 1 in combination with an indwelling medical device.

39. The adjunctive accessory article of claim 38 wherein said indwelling medical device comprises a catheter.

40. The adjunctive accessory article of claim 38 wherein said indwelling medical device comprises a stent.

41. The adjunctive accessory article of claim 38 wherein said indwelling medical device comprises an endourethral device.

42. A modular medicament delivery article for controlled release of at least a single physiologically active agent from an indwelling medical device so equipped, said article comprising a polymeric active agent capsule and an active agent contained therein, said capsule having an end portion adapted for receipt and retention within an aperture of the indwelling medical device.
